(19) United States
(12) Patent Application Publication
(10) Pub. No.: US 2024/0165274 A1
(43) Pub. Date: May 23, 2024

(54) METHODS OF DETECTING SYSTEMIC AMYLOIDOSIS VIA BINDING TO MISFOLDED OR AGGREGATED PROTEIN

(71) Applicant: Amydis, Inc., Los Angeles, CA (US)

(72) Inventors: Stella T. Sarraf, Beverly Hills, CA (US); Peter W. Vanderklish, San Diego, CA (US); Ali Tafreshi, Los Angeles, CA (US)

(21) Appl. No.: 18/550,099

(22) PCT Filed: Mar. 11, 2022
(86) PCT No.: PCT/US2022/020013

§ 371 Date: Sep. 11, 2023

Related U.S. Application Data
(60) Provisional application No. 63/160,602, filed on Mar. 12, 2021.

(51) Int. Cl. A61K 49/00; G01N 33/68

(52) U.S. Cl. CPC A61K 49/0021; G01N 33/6893

(57) ABSTRACT

Provided herein are methods and compositions for determining whether a patient suffers from systemic amyloidosis, comprising detecting the presence of a misfolded or aggregated protein in a tissue of the patient, wherein the detecting comprises contacting the misfolded or aggregated transthyretin protein with a compound described herein. In some embodiments, the misfolded or aggregated protein is transthyretin.

### CROSS-REFERENCE TO RELATED APPLICATIONS

This application claims the benefit under 35 U.S.C. § 119(e) of U.S. Provisional Application No. 63/160,602, filed Mar. 12, 2021, which is hereby incorporated by reference in its entirety.

### BACKGROUND

Systemic amyloidosis is a disease that occurs when an abnormal protein, called amyloid, builds up in organs and interferes with their normal function. Amyloid can be formed from any of a number of different types of protein. Organs that may be affected include the heart, kidneys, liver, spleen, nervous system and digestive tract. Some varieties of amyloidosis may lead to life-threatening organ failure. A patient may not experience signs and symptoms of amyloidosis until the condition is advanced.

Transthyretin is a protein made by the liver that helps carry thyroid hormone and vitamin A in the blood. Misfolding and aggregation of transthyretin (TTR) is associated with a number of conditions. Familial amyloidotic polyneuropathy (FAP), also referred to as transthyretin amyloid polyneuropathy, is a rare, inherited, and progressive disease caused by the abnormal deposits of proteins or amyloids around peripheral nerves and other tissues. FAP is caused by mutations in the TTR gene that destabilize the TTR tetramer. FAP was first identified in 1952, when it was observed in several families in Portugal. In some areas of northern Portugal, FAP affects about 1 in 500 people, and occurs throughout the world. In some instances, liver transplant is indicated. There is currently no cure for FAP.

Another important disorder is a progressive systemic disorder called amyloid transthyretin (ATTR) amyloidosis. In ATTR amyloidosis, the protein deposits in the heart and/or the nerves and other organs and tissues. ATTR amyloidosis is a slowly progressive condition characterized by the buildup of abnormal deposits of a protein called amyloid (amyloidosis) in the body's organs and tissues.

There is a need for safe and convenient methods for detecting systemic amyloidosis.

### SUMMARY

In some embodiments, a method for determining whether a patient suffers from systemic amyloidosis, for example, familial amyloidotic polyneuropathy (FAP) or transthyretin (ATTR) amyloidosis, is provided, comprising administering to an eye of the subject a compound described herein, or a pharmaceutically acceptable salt thereof, and detecting the presence or absence of a misfolded or aggregated protein. The misfolded or aggregated protein may be a misfolded or aggregated transthyretin protein (TTR). The compound may be a compound of formula I or may be any compound described herein. In certain embodiments, an amyloid is detected, wherein the amyloid does not comprise amyloid beta peptide.

In some embodiments, a method for diagnosing systemic amyloidosis is provided, comprising administering to an eye of a subject in need thereof a compound described herein, or a pharmaceutically acceptable salt thereof, and detecting the presence or absence of a misfolded or aggregated transthyretin protein.

In some embodiments, provided herein is a compound having a following structure:

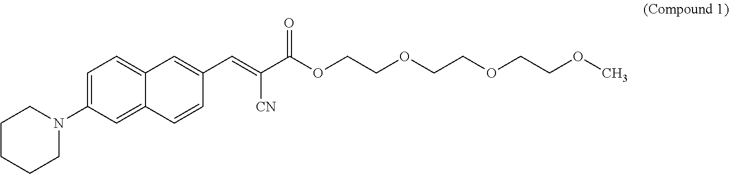 [SMILES: COCCOCCOCCOC(=O)/C(C#N)=C/c1ccc2cc(N3CCCCC3)ccc2c1]

or a pharmaceutically acceptable salt thereof. In some embodiments, provided herein is Compound 1 for use in diagnosing systemic amyloidosis. In some embodiments, provided herein is Compound 1 for use in determining whether a patient suffers from systemic amyloidosis. In some embodiments, provided herein is Compound 1 for use in preparing a patient for diagnosis of systemic amyloidosis, wherein the Compound 1 is topically administered to an eye of the patient.

The instant disclosure provides simple and noninvasive methods for detecting systemic amyloidosis by applying amyloid-binding compounds to the surface of an eye of the subject and directly detecting fluorescence produced thereby. In ATTR and other systemic amyloidoses, amyloid formation is initiated by dissociation of the TTR tetramer to form an aggregation-prone monomer that self-associates to form small oligomers, amorphous aggregates, and/or fibrils. On the other hand, amyloid beta comprises peptides of 36-43 amino acids which are cleaved from amyloid precursor protein. Both mutant and wild-type TTR forms aggregates and fibrils, and formation compound through complex mechanisms that are poorly understood. Accordingly, the instant disclosure provides surprisingly allows systemic amyloidosis to be diagnosed by surface detection of TTR amyloids by applying certain compounds to the surface of the eye for fluorescence detection thereof.

### BRIEF DESCRIPTION OF THE DRAWINGS

FIG. 1 illustrates fluorescence spectra of Compound 1 with and without the presence of aggregated TTR.

FIG. 2 illustrates the relationship between fluorescence intensity and the concentration of Compound 1 in presence of aggregated TTR at a constant concentration.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

The term “alkyl,” by itself or as part of another substituent, means, unless otherwise stated, a straight (i.e. unbranched) or branched chain, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene” by itself or as part of another substituent means a divalent radical derived from an alkyl, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S. The nitrogen and sulfur atoms may optionally be oxidized, and the nitrogen heteroatom may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, CH═CH—N(CH₃)—CH₃, O—CH₃, —O—CH-₂-CH₃, and —CN. Up to two heteroatoms may be consecutive, such as, for example, —CH₂—NH—OCH₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. R′ and R″ are each as defined below with respect to “substituents.”

The terms “cycloalkyl” and “heterocycloalkyl,” by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, fluoromethyl, difluoromethyl, trifluoromethyl, 2,2,2-trifluoroethyl, 4-chlorobutyl, 3-bromopropyl, and the like.

The term “acyl” means C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl or substituted or unsubstituted heteroaryl.

The term “aryl” means, unless otherwise stated, a polyunsaturated, aromatic, hydrocarbon substituent which can be a single ring or multiple rings (preferably from 1 to 3 rings) which are fused together (i.e. a fused ring aryl) or linked covalently. A fused ring aryl refers to multiple rings fused together and at least one of the fused rings is an aryl ring. The term “heteroaryl” refers to aryl groups (or rings) that contain from one to four heteroatoms selected from N, O, and S. The nitrogen and sulfur atoms are optionally oxidized, and the nitrogen atom(s) are optionally quaternized. Thus, the term “heteroaryl” includes fused ring heteroaryl groups (i.e. multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent means a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxyl)propyl, and the like).

The term “oxo” as used herein means oxygen that is double bonded to a carbon atom.

The term “alkylsulfonyl” as used herein means a moiety having the formula —S(O₂)—R′, where R′ is an alkyl group as defined above. R′ may have a specified number of carbons (e.g. “C₁-C₄ alkylsulfonyl”).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m(1) where m′ is the total number of carbon atoms in such radical. R′, R″, R′″ and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound disclosed herein includes more than one R group, for example, each of the R groups is independently selected as are each R′, R″, R′ and R′″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound disclosed herein includes more than one R group, for example, each of the R groups is independently selected as are each R′, R″, R′ and R″″ groups when more than one of these groups is present.

Two of the substituents on adjacent atoms of the aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or S(O)₂NR′—. The substituents R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “pharmaceutically acceptable salts” is meant to include salts of the active compounds which are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds disclosed herein contain both basic and acidic functionalities that allow the compounds to be converted into either base or acid addition salts.

Thus, the compounds disclosed herein may exist as salts, such as with pharmaceutically acceptable acids. Examples of such salts include hydrochlorides, hydrobromides, sulfates, methanesulfonates, nitrates, maleates, acetates, citrates, fumarates, tartrates (e.g., (+)-tartrates, (−)-tartrates or mixtures thereof including racemic mixtures), succinates, benzoates and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds of Formula I in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compounds described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

Misfolded or Aggregated Protein

Some systemic amyloidoses, and familial amyloidotic polyneuropathy (FAP) and ATTR amyloidosis in particular, are associated with release and/or accumulation of misfolded transthyretin protein. The misfolded or aggregated transthyretin protein may be detected by contacting with a compound as described herein, wherein the contacting, upon activation by a light, causes emission of detectable signal. Generally, the compositions and methods described herein are useful for detection of misfolded or aggregated transthyretin protein in any tissue of the patient. However, the misfolded or aggregated transthyretin protein may accumulate in the eye of a patient. In particular the misfolded or aggregated transthyretin protein may accumulate in the conjunctiva of the eye. Such presence of misfolded or aggregated transthyretin protein in the retina can be detected with compounds that bind to the misfolded or aggregated transthyretin protein, which binding can then be detected by means such as laser-activated fluorescence scanning of the retina.

Transthyretin (“TTR”, “TTR protein” or “TBPA”) is a transport protein in the serum and cerebrospinal fluid that carries the thyroid hormone thyroxine (T4) and retinol-binding protein bound to retinol. The liver secretes transthyretin into the blood, and the choroid plexus secretes TTR into the cerebrospinal fluid. TTR may also be referred to as prealbumin or thyroxine-binding prealbumin.

TTR is a unique human protein synthesized in hepatocytes, retinal pigment epithelial cells, choroid plexus epithelium, pancreatic α-cells, Schwann cells, and neurons under some conditions. It is a carrier of retinol binding protein (RBP) charged with retinol in serum and a minor carrier of the thyroid hormone precursor thyroxine (T4) prior to its conversion to the physiologically more active tri-iodo-thyronine (T3) by tissue deiodinases. Only a small fraction of the circulating TTR carries T4 while 25-50% is loaded with RBP-retinol. However, in cerebrospinal fluid choroid plexus synthesized TTR is the major T4 carrier. The crystal structure shows a twofold axis of symmetry. It is assembled as a dimer of dimers around a central channel, which is primarily hydrophobic and contains the two T4 binding sites. T4 binding in the first site induces an allosteric change that makes the second site less accessible to its natural ligand. While different portions of the protein bind T4 and RBP, both stabilize the tetrameric structure reducing its tendency to dissociate.

TTR is encoded by a single gene on chromosome 18 that encompasses approximately 19 Kb of DNA with 4 exons included within 7 Kb, 6 Kb of upstream (5′) sequence and 6 Kb downstream containing the conventional 3′ non-coding sequence that allows normal mRNA processing after transcription. The promoter proximal 2 Kb appears to contain all the sequences required for tissue specific expression of the gene. Transthyretin isoform sequences include NP_000362.1. The transthyretin sequence may be a variant or a mutant. The variant or mutant may be any known form, including but not limited to the following:
G6S, C10R, L12P, M13I, D18N, D18G, D18E, A19D, V20I, R21Q, S23N, P24S, A25S, A25T, V28M, V28S, V30M, V30A, V30G, V30L, V32A, V32G, F33I, F33L, F33V. F33C, R34G, R34T, K35N, K35T, A36D, A36P, D38A, D38V, D39V. W41L, E42G, E42D, F44Y, F44S, F44L, A45S, A45T, A45D, A45G, G47R, G47A, G47E, G47V, T49A, T49P, T49I, T49S, S50R, S50I, E51G, S52P, G53R, G53E, G53A, E54L, E54K, E54G, E54D, E54Q, L55Q, L55R, L55P, H56R, G57R, L58R, L58H, T59R, T59K, T60A, E61K, E61G, E62K, F64I, F64L, F64S, G67R, G67E, I68L, Y69H, Y69I, K70N, V71A, E72G, I73V, D74H, S77F, S77Y, Y78F, A81T, A81V, G83R, I84N, I84S, I84T, H88R, E89Q, E89K, H90N, H90D, A91S, E92K, V93M, V94A, A97S, A97G, G101S, P102R, R103S, R104C, R104H, I107V, I107F, I107M, A109S, A109T, A109V, L111M, S112I, P113T, Y114C, Y114H, Y114S, Y116S, T119M, A120S, V122A, V122I, P125S.

TTR is a non-disulfide linked homo-tetramer in which the mature polypeptide monomer, after cleavage of the leader sequence, contains 127 amino acids. The tetramer is generally stable with Ka=1.1×10²⁴ M⁻³. Dissociation of the TTR tetramer may lead to deposition of the protein in tissue, and amyloid formation. The misfolded or aggregated TTR protein may be an N-oligomer. TTR may be wild-type TTR, or may be a variant or a mutant thereof.

In some embodiments, the misfolded or aggregated protein may be beta2 macroblobulin, leukocyte chemotactic factor 2, Apolipoprotein AIV, Apolipoprotein CII, Apolipoprotein CIII, or lysozyme, or a mutant or a variant thereof, each of which is known in the art. The misfolded or aggregated protein may be an amyloid thereof.

In accordance with some embodiments of the present disclosure, therefore, provided is a method for determining whether a patient suffers from a systemic amyloidosis. The method entails detecting the presence of a misfolded or aggregated transthyretin protein in a tissue such as an eye of a subject, by contacting a tissue of the subject with a compound described herein. The contacting may be in vivo or ex vivo. The contacting may be by topical administration. It is contemplated that the accumulation of the misfolded or aggregated transthyretin protein may occur in the conjunctiva of the eye. Accordingly, the detection can target misfolded or aggregated transthyretin protein in the conjunctiva by topical administration to the surface of the eye. When administration is topical, the compound may be formulated in an ophthalmologically acceptable formulation.

In another embodiment, provided is a method for preparing a patient for diagnosis of a systemic amyloidosis, in particular familial amyloidotic polyneuropathy (FAP) or ATTR amyloidosis, which method comprises administering to the patient a compound that specifically binds a misfolded or aggregated transthyretin protein. The compound may be administered to an eye of the patient. Once the compound is administered to the patient, its binding to the misfolded or aggregated transthyretin protein may be detected with any method, including those methods described herein, which binding indicates accumulation of the misfolded transthyretin protein, an indication of a systemic amyloidosis.

Misfolded or Aggregated Protein Binding Compounds

The present disclosure further provides a compound capable of binding to a misfolded or aggregated protein. In some embodiments, the compound is capable of binding to a misfolded or aggregated transthyretin protein. In one embodiment, the compound may be selected from those compounds described in International Publication No. WO 2011/072257, or U.S. Pat. No. 9,551,722 which are incorporated by reference in their entirety. Synthesis of the compounds may proceed by methods known in the art including those described in the incorporated references.

In some embodiments, the disclosure provides a compound of Formula I or a salt or solvate thereof.

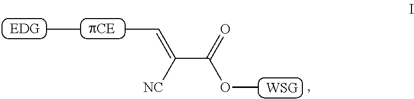

wherein
EDG is:
R¹-substituted or unsubstituted alkyl, R¹-substituted or unsubstituted cycloalkyl, R¹-substituted or unsubstituted heteroalkyl, R¹-substituted or unsubstituted heterocycloalkyl, R¹-substituted or unsubstituted aryl, R¹-substituted or unsubstituted heteroaryl, OR²NR⁴C(O)R³, —NR⁴R⁵, —SR⁶, or PR⁷R⁸,
wherein
   R¹ is halogen, —OR⁹, —NR¹⁰R¹¹, unsubstituted alkyl, unsubstituted heteroalkyl, unsubstituted cycloalkyl, unsubstituted heterocycloalkyl, unsubstituted aryl, or unsubstituted heteroaryl;
R², R³, R⁴, R⁵, R⁶, R⁷ and R⁸ are independently hydrogen, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl or R¹²-substituted or unsubstituted heteroaryl, wherein R⁴ and R⁵ are optionally joined together to form an R¹²-substituted or unsubstituted heterocycloalkyl, or R¹²-substituted or unsubstituted heteroaryl;
R⁹ and R¹⁰ are independently hydrogen, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl, or R¹²-substituted or unsubstituted heteroaryl, wherein R¹⁰ and are optionally joined together to form an R¹²-substituted or unsubstituted heterocycloalkyl, or R¹²-substituted or unsubstituted heteroaryl;
R¹² is halogen, —OR¹³, —NR¹⁴R¹⁵, R¹⁶-substituted or unsubstituted alkyl, R¹⁶-substituted or unsubstituted heteroalkyl, R¹⁶-substituted or unsubstituted cycloalkyl, R¹⁶-substituted or unsubstituted heterocycloalkyl, R¹⁶-substituted or unsubstituted aryl, or R¹⁶-substituted or unsubstituted heteroaryl;
   R¹³, R¹⁴ and R¹⁵ are independently hydrogen or unsubstituted alkyl; and
   R¹⁶ is unsubstituted alkyl, unsubstituted heteroalkyl, unsubstituted cycloalkyl, unsubstituted heterocycloalkyl, unsubstituted aryl, or unsubstituted heteroaryl; wherein
πCE has the formula:

-L¹-(A¹)_{q}-L²-(A²)ᵣ-L³- or-L¹-(A)_{q}-L⁴-A³-L²-(A²)ᵣ-L³-,
wherein
q and r are independently 0 or 1, wherein at least one of q or r is 1;
A¹, A² and A³ are independently R¹⁷-substituted or unsubstituted arylene or R⁷-substituted or unsubstituted heteroarylene;
L¹, L², L³ and L4 are independently a bond or a linking group having the formula:

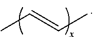

wherein x is an integer from 1 to 50;
R¹⁷ is halogen, —OR¹⁸, —NR¹⁹R²⁰, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl;
   R¹⁸, R¹⁹ and R²⁰ are independently hydrogen, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl;
R²¹ is halogen, —OR²², —NR²³R²⁴, unsubstituted alkyl, unsubstituted heteroalkyl, unsubstituted cycloalkyl, unsubstituted heterocycloalkyl, unsubstituted aryl, or unsubstituted heteroaryl;
   R²², R²³ and R²⁴ are independently hydrogen or unsubstituted alkyl; and
wherein
WSG is R²⁵-substituted or unsubstituted alkyl, R²⁵-substituted or unsubstituted heteroalkyl, R²⁵-substituted or unsubstituted cycloalkyl, R²⁵-substituted or unsubstituted heterocycloalkyl, R²⁵-substituted or unsubstituted aryl, R²⁵-substituted or unsubstituted heteroaryl;
wherein
   R²⁵ is halogen, —OR²⁶, —NR²⁷R²⁸, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl;
   R²⁶, R²⁷ and R²⁸ are independently hydrogen, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl, wherein R²⁷ and R²⁸ are optionally joined together to form an R²⁹-substituted or unsubstituted heterocycloalkyl, or R²⁹-substituted or unsubstituted heteroaryl;
   R²⁹ is halogen, —OR³⁰, —NR³¹R³², unsubstituted alkyl, unsubstituted heteroalkyl, unsubstituted cycloalkyl, unsubstituted heterocycloalkyl, unsubstituted aryl, or unsubstituted heteroaryl; and
   R³⁰, R³¹ and R³² are independently hydrogen or unsubstituted alkyl;

In certain embodiments, the compound is

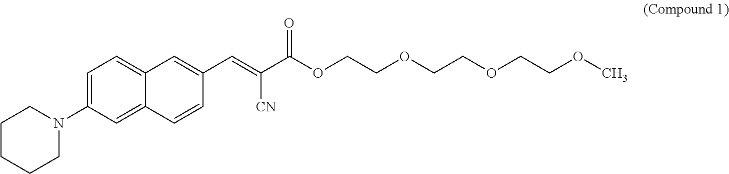 [SMILES: COCCOCCOCCOC(=O)/C(C#N)=C/c1ccc2cc(N3CCCCC3)ccc2c1]

or a pharmaceutically acceptable salt thereof.

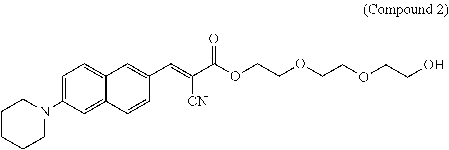 [SMILES: N#C/C(=C\c1ccc2cc(N3CCCCC3)ccc2c1)C(=O)OCCOCCOCCO]

or a pharmaceutically acceptable salt thereof.

Diseases and Disorders and Treatment Thereof

Provided herein are methods for determining whether a patient suffers from a disease or disorder, comprising detecting the presence of a misfolded or aggregated protein in an eye of the subject, wherein the detecting comprises contacting the misfolded or aggregated protein with a compound described herein. The misfolded or aggregated protein may be a misfolded or aggregated transthyretin protein. The disease or disorder may be a systemic amyloidosis. The systemic amyloidosis may be familial amyloidotic polyneuropathy (FAP). The systemic amyloidosis may be ATTR amyloidosis. The compound may be a compound described herein. The contacting may be in vivo. The tissue may be an eye tissue. The tissue may be a wrist, spinal canal, heart, or lacrimal gland.

Symptoms of systemic amyloidosis may include: swelling of ankles and/or legs, severe fatigue, weakness, shortness of breath, numbness, tingling or pain in hands or feet, pain in the wrist (carpal tunnel syndrome), diarrhea, bloody stool, constipation, unintentional weight loss (e.g., of more than 10 pounds (4.5 kilograms)), enlarged tongue, rippled tongue, skin thickening, bruising, purplish patches around the eyes, irregular heartbeat, and/or difficulty swallowing.

The systemic amyloidosis may be familial amyloidotic polyneuropathy (FAP), ATTR amyloidosis, TTR cardiac amyloidosis, TTR amyloid cardiomyopathy (ATTR-CM), familial or hereditary ATTR amyloidosis (ATTRv or ATTRm), senile systemic amyloidosis (SSA or ATTRwt), AL amyloidosis, AA (serum amyloid A amyloidosis), AA amyloidosis resulting from inflammation due to infection, rheumatological disease, autoinflammatory and autoimmune diseases whether acquired or hereditary, cancer or neoplasms, Fibrinogen alpha-chain amyloidosis, Apolipoprotein A-1 and A-2 Amyloidoses, Gelsolin amyloidosis, Ab2M (beta2 macroblobulin) amyloidosis, ALECT2 (leukocyte chemotactic factor 2) Amyloidosis, AApoAIV (Apolipoprotein AIV) amyloidosis, AApoCII (Apolipoprotein CII) amyloidosis, AApoCIII (Apolipoprotein CIII) amyloidosis, ALys (lysozyme) amyloidosis.

In some embodiments, the systemic amyloidosis is familial amyloid polyneuropathy (TTR-FAP or FAP). FAP, also referred to as transthyretin amyloid polyneuropathy, is a rare, inherited, and progressive disease caused by the abnormal deposits of proteins or amyloids around peripheral nerves and other tissues. FAP is caused by mutations in the TTR gene that destabilize the TTR tetramer. To date, more than 100 different mutations in the gene have been reported. The symptoms of FAP include progressive sensorimotor and autonomic neuropathy, including peripheral neuropathy that may appear early as an abnormal sensation in the legs and feet, such as numbness, tingling, or burning, and autonomic neuropathy, when the nerves that control involuntary bodily functions such as blood pressure, temperature control, and digestion, are damaged. Degeneration of motor fibers may cause progressive weakness and gait disturbances. One treatment of FAP is liver transplantation. Other treatments include gene therapy, immunization, dissolution of TTR aggregates, free radical scavengers, and administration of meglumine.

In some embodiments, the systemic amyloidosis is AL amyloidosis (immunoglobulin light chain amyloidosis). The most common type of amyloidosis in developed countries, AL amyloidosis is also called primary amyloidosis. It usually affects the heart, kidneys, liver and nerves.

In some embodiments, the systemic amyloidosis is AA amyloidosis. Also known as inflammatory amyloidosis, this variety is usually triggered by an inflammatory disease, such as rheumatoid arthritis. Treatments for severe inflammatory conditions may treat or prevent AA amyloidosis. It commonly affects the kidneys, liver and spleen.

In some embodiments, the systemic amyloidosis is localized amyloidosis. This type of amyloidosis often has a better prognosis than the varieties that affect multiple organ systems. Typical sites for localized amyloidosis include the bladder, skin, throat or lungs. Correct diagnosis is important so that treatments that affect the entire body can be avoided.

In some embodiments, the systemic amyloidosis is the systemic amyloidosis is ATTR amyloidosis. The ATTR amyloidosis may be TTR Cardiac Amyloidosis, TTR Amyloid Cardiomyopathy (ATTR-CM), familial or hereditary ATTR amyloidosis (ATTRv or ATTRm), or senile systemic amyloidosis (ATTRwt).

ATTRm amyloidosis is a multi-system disorder with cardiovascular, peripheral and autonomic nerve involvement that can be difficult to diagnose due to phenotypic heterogeneity. ATTRm amyloidosis is a rare disease with diverse clinical manifestations that is in part determined by the genotype. Given this complexity, there can be a delay in diagnosis of up to 4 years from symptom onset for patients with ATTRm presenting with a peripheral neuropathy and up to 8 years for patients presenting with a cardiomyopathy. Carpal tunnel syndrome (CTS) can be the initial symptom in up to 33% of patients with a mean period of 4-6 years before other organs are clinically involved. Patients then usually develop a peripheral and autonomic neuropathy, and often cardiac involvement. As TTR is also produced within the choroid plexus and the epithelium of the retina, central nervous system (CNS) manifestations can also rarely occur, and are more common for some disease-causing mutations that have a predilection for the CNS, such as ATTRL12P. Additional clinical manifestations include peripheral neuropathy and autonomic neuropathy. V30M is the most common ATTR mutation, but other mutations may be underlying including ATTRV122I and ATTRL12P, or a variant or a mutant as described herein.

Amyloidogenic protein variants causing the autosomal dominant clinical disorders Familial Amyloidotic Polyneuropathy (a sensori-motor and autonomic polyneuropathy) and Familial Amyloidotic Cardiomyopathy have been found in 77 of the 127 amino acids in TTR protein. Forty residues have been found to have a single amyloidogenic mutation while fifteen have 2, six have 3, five have 4, and one has 5. Fifty amino acids have none and 12 mutations have been described that did not lead to clinically detectable amyloidosis, although two of the involved residues had both amyloidogenic and non-amyloidogenic substitutions. There is an increasing frequency of wild type TTR amyloid deposition in the heart, carpal tunnel and gut associated with increasing age currently thought to be related to post-synthetic (perhaps oxidative) changes that may render the wild type protein less stable although other, as yet undefined, mechanisms may be responsible. In the case of the mutations it appears that they all form tetramers which are kinetically or thermodynamically unstable under physiologic conditions resulting in enhanced dissociation releasing monomers which are susceptible to rapid misfolding, aggregation, and fibril formation. These observations suggest that the monomers functionally “chaperone” each other.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) ameliorating, slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. In some embodiments, the methods provide for administration of a compound described herein to a patient (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of a systemic amyloidosis. The therapeutically effective amount may vary depending on the subject, and disease or condition being treated, the weight and age of the patient, the severity of the disease or condition, and the manner of administering, which can readily be determined by one or ordinary skill in the applicable art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human patients. Such properties may be examined using commonly known methods to those skilled in the art.

In some embodiments, the misfolded or aggregated protein is not amyloid beta (AP) peptide, α-Synuclein, prion peptide, huntingtin, serum amyloid A, lysozyme, amylin, immunoglobulin light chain, semen derived enhancer of viral infection, PAB, SEM1, protegrin-1, CsgA-R5, and CsgA-R1, superoxide dismutase, insulin, or p53. In some embodiments, the misfolded or aggregated protein does not comprise amyloid beta (Aβ) peptide. In some embodiments, the disease or disorder is not pre-eclampsia.

Detection of Misfolded or Aggregated Protein

Provided herein are methods for diagnosis of a disease or disorder, comprising topically administering to an eye of the subject a compound described herein. The disease or disorder may be a systemic amyloidosis. The systemic amyloidosis may be familial amyloidotic polyneuropathy (FAP). The systemic amyloidosis may be ATTR amyloidosis. The method may comprise detecting a binding of the compound to a misfolded or aggregated protein. The method may comprise detecting a binding of the compound to a misfolded or aggregated transthyretin protein. The binding, upon activation by a light, may cause emission of a detectable signal. The signal may be a fluorescent or infrared signal. The administration may be topical administration and/or may be localized at the conjunctiva of the eye.

Detection of a misfolded or aggregated protein can be made with a compound that can selectively bind to the misfolded or aggregated protein. In one embodiment, the compound, when bound to a misfolded or aggregated protein, can be detected through its emitted fluorescent signal, upon activation by a laser light.

In situ detection of binding of a protein-binding probe, for example, a compound described herein, to a misfolded or aggregated protein in the eye of the patient can be facilitated with an imaging device. The imaging device may be handheld or portable. The imaging device can include a lens and an image sensor, and optionally a laser light source. When the light source emits laser light to the tissue, for example, the conjunctiva, if misfolded or aggregated protein is accumulated there and has bound to a compound that fluoresces when bound, the accumulation can be readily detected and quantitated by the lens and image sensor that collects and senses a fluorescent signal.

The methods for determining whether a patient suffers from a disease or disorder such as systemic amyloidosis, may be conducted in any manner described herein or as known in the art. In some embodiments, the contacting, upon activation by a light, causes emission of detectable signal. In some embodiments, the detectable signal is a fluorescent signal. In some embodiments, the misfolded or aggregated protein is present in the tissue as fibrils. In some embodiments, the misfolded or aggregated protein is present in the tissue as plaques. In some embodiments, the method does not comprise determining fluorescence decay. In some embodiments, the method does not comprise determining fluorescence decay based on comparison to a reference.

In some embodiments, provided is a method for monitoring response to a treatment of a subject having a systemic amyloidosis, comprising: (i) forming a detectable complex by contacting an effective amount of a compound described herein with an eye of the subject; and (ii) detecting the formation of the detectable complex, wherein a decrease of detectable complex as compared to before the treatment indicates that the subject is responsive to the treatment. In some embodiments, the compound is applied topically to the eye.

Administration and Pharmaceutical Compositions

In some embodiments, a pharmaceutical composition of a compound described herein is administered to an eye of a subject. In some embodiments, the compound is delivered to the ocular surface. In some embodiments, the compound is administered as an eye drop. The administration may be topical administration.

The compound may be effective over a wide dosage range. In some embodiments, in the application to adult humans, dosages from 0.01 to 1000 mg, from 0.5 to 100 mg, from 1 to 50 mg per day, and from 5 to 40 mg per day are examples of dosages that are used. An exemplary dosage is 10 to 30 mg per day. In the applications to juveniles, the dosage may be the same or less than the adult dose. In some embodiments the effective amount of the compound corresponds to about 50-500 mg of compound per adult patient. The exact dosage will depend upon the route of administration, the form in which the compound is administered, the patient to be treated, the body weight of the patient to be treated, and the preference and experience of the attending physician.

In some embodiments the effective amount of the compound corresponds to about 0.01-1000 mg of compound per human patient per dosage. In some embodiments, the effective dose of compound is 50-500 mg per human per dosage. In some embodiments the effective amount corresponds to about 0.01-100 mg, 0.01-200 mg, 0.01-300 mg, 0.01-400 mg, 0.01-500 mg, 0.01-600 mg, 0.01-700 mg, 0.01-800 mg, 0.01-900 mg, 0.01-1000 mg, 0.1-100 mg, 0.1-200 mg, 0.1-300 mg, 0.1-400, 0.1-500 mg, 0.1-600 mg, 0.1-700 mg, 0.1-800 mg, 0.1-900 mg, 0.1-1000 mg, 1-100 mg, 1-200 mg, 1-300 mg, 1-400 mg, 1-500 mg, 1-600 mg, 1-700 mg, 1-800 mg, 1-900 mg, 100-200 mg, 100-300 mg, 100-400 mg, 100-500 mg, 100-600 mg, 100-700 mg, 100-800 mg, 100-900 mg, 100-1000 mg, 200-300 mg, 200-400 mg, 200-500 mg, 200-600 mg, 200-700 mg, 200-800 mg, 200-900 mg, 200-1000 mg, 300-400 mg, 300-500 mg, 300-600 mg, 300-700 mg, 300-800 mg, 300-900 mg, 300-1000 mg, 400-500 mg, 400-600 mg, 400-700 mg, 400-800 mg, 400-900 mg, 400-1000 mg, 500-600 mg, 500-700 mg, 500-800 mg, 500-900 mg, 500-1000 mg, 600-700 mg, 600-800 mg, 600-900 mg, 600-1000 mg, 700-800 mg, 700-900 mg, 700-1000 mg, 800-900 mg, 800-1000 mg or about 900-1000 mg per human per dosage. In some embodiments, the effective amount corresponds to about 50-100 mg, 50-400 mg, 50-500 mg, 100-200 mg, 100-300 mg, 100-400 mg, 100-500 mg, 200-300 mg, 200-400 mg, 200-500, 300-400 mg, 300-500 mg, or 400-500 mg per adult human per dosage.

In some embodiments, the compound is administered in a single dose. In some embodiments, the compound is administered in multiple doses. In some embodiments, dosing is about once, twice, three times, four times, five times, six times, or more than six times per day. In some embodiments, dosing is about once a month, once every two weeks, once a week, or once every other day. In another case the compound and another agent are administered together about once per day to about 6 times per day. In some embodiments the administration of the compound and an agent continues for less than about 7 days. In yet another case the administration continues for more than about 6, 10, 14, 28 days, two months, six months, or one year. In some embodiments, continuous dosing is achieved and maintained as long as necessary.

In some embodiments, the compound is administered one to ten times, one to four times, or once a day. In some embodiments, the compound is administered 1, 2, 3, 4, 5, 6, 7, 8, 9, or 10 times a day. In some embodiments, the compound is administered as drops. In some embodiments, the size of the drop administered is in the range of about 10-100 μL, about 10-90 μL, about 10-80 μL, about 10-70 μL, about 10-60 μL, about 10-50 μL, about 10-40 μL, about 10-30 μL, about 20-100 μL, about 20-90 μL, about 20-80 μL, about 20-70 μL, about 20-60 μL, about 20-50 μL, about 20-40 μL, or about 20-30 μL. One example of the disclosure administers a drop in the range of about 10 to about 30 μL. One example of the disclosure administers a drop in the range of about 10 to about 100 μL. One example of the disclosure administers a drop in the range of about 20 to about 50 μL. One example of the disclosure administers a drop in the range of about 20 to about 40 μL. One example of the disclosure administers a drop in the range of about 10 to about 60 μL. In some embodiments, the eye formulations of the disclosure is administered several drops per time, for example 1-3 drops per time, 1-3 drops per time, 1-4 drops per time, 1-5 drops per time, 1-6 drops per time, 1-7 drops per time, 1-8 drops per time, 1-9 drops per time, 1-10 drops per time, 3-4 drops per time, 3-5 drops per time, 3-6 drops per time, 3-7 drops per time, 3-8 drops per time, 3-9 drops per time, 3-10 drops per time, 5-6 drops per time, 5-7 drops per time, 5-8 drops per time, 5-9 drops per time, 5-10 drops per time, 7-8 drops per time, 7-9 drops per time or 9-10 drops per time. In one example, the formulations of the disclosure are administered about one drop per time and 1-6 times per day.

Pharmaceutical Compositions/Formulations

In some embodiments, the compound described herein is formulated into a pharmaceutical composition. In some embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound as described herein and a pharmaceutically acceptable diluent(s), excipient(s), or carrier(s). In certain cases, the compound described herein is administered as pharmaceutical compositions in which one or more compounds, are mixed with other active ingredients, as in combination therapy. In specific cases, the pharmaceutical compositions include one or more compounds as described herein.

A pharmaceutical composition, as used herein, refers to a mixture of a compound described herein, with other chemical components, such as carriers, stabilizers, diluents, dispersing agents, suspending agents, thickening agents, and/or excipients. In certain cases, the pharmaceutical composition facilitates administration of the compound to an organism. In some embodiments for practicing the methods of treatment or use provided herein, therapeutically effective amounts of one or more compounds described herein are administered in a pharmaceutical composition to a mammal having a disease or condition to be detected, diagnosed or treated. In specific cases, the mammal is a human. In certain cases, therapeutically effective amounts vary depending on the severity of the disease, the age and relative health of the patient, the potency of the compound used and other factors. The compounds described herein are used singly or in combination with one or more therapeutic agents as components of mixtures.

In some embodiments, the one or more compounds is formulated in an aqueous solution. In specific cases, the aqueous solution is selected from, by way of example only, a physiologically compatible buffer, such as Hank's solution, Ringer's solution, aqueous acetate buffer, aqueous citrate buffer, aqueous carbonate buffer, aqueous phosphate buffer or physiological saline buffer.

In some embodiments, the compounds described herein are formulated for ocular administration. In some embodiments, the ocular formulations is liquid (in form of solutions, suspensions, powder for reconstitution, sol to gel systems), semi solids (ointments and gels), solids (ocular inserts), and intraocular dosage forms (injections, irrigating solutions and implants). The compound can be formulated for topical administration, e.g., to the eye.

Provided herein are ophthalmic formulations comprising the compounds described herein and an ophthalmologically acceptable component. The ophthalmic formulation may be administered in any form suitable for ocular drug administration, e.g., as a solution, suspension, ointment, gel, liposomal dispersion, colloidal microparticle suspension, or the like, or in an ocular insert, e.g., in an optionally biodegradable controlled release polymeric matrix.

By a “pharmaceutically acceptable” or “ophthalmologically acceptable” component is meant a component that is not biologically or otherwise undesirable, i.e., the component may be incorporated into an ophthalmic formulation of the disclosure and administered topically to a patient's eye without causing any undesirable biological effects or interacting in a deleterious manner with any of the other components of the formulation composition in which it is contained. When the term “pharmaceutically acceptable” is used to refer to a component other than a pharmacologically active agent, it is implied that the component has met the required standards of toxicological and manufacturing testing or that it is included on the Inactive Ingredient Guide prepared by the U.S. Food and Drug Administration. See. e.g., Kaur et al., Drug Development and Industrial Pharmacy (2002) 28(5), 473-493.

Ophthalmic formulations may be adapted for topical administration to the eye in the form of a suspension or emulsion. The ophthalmic formulation may include an ophthalmologically acceptable carrier. Such carriers include, for example, water, mixtures of water, for example, phosphate buffer, boric acid, sodium chloride, and sodium borate, and water-miscible solvents such as lower alcohols, aryl alcohols, polyalkylene glycols, carboxymethylcellulose, polyvinylpyrrolidone, and isopropyl myristate. The ophthalmic formulation may also include one or more excipients such as emulsifying agents, preserving agents, wetting agents, bodying agents. For example, the ophthalmic formulation may include polyethylene glycols 200, 300, 400 and 600, carbowaxes 1,000, 1,500, 4,000, 6,000 and 10,000, antibacterial components such as quaternary ammonium compounds, phenylmercuric salts, thimerosal, methyl and propyl paraben, benzyl alcohol, phenyl ethanol, buffering agents such as sodium borate, sodium acetates, gluconate buffers, and other agents such as sorbitan monolaurate, triethanolamine, oleate, polyoxyethylene sorbitan monopalmitylate, dioctyl sodium sulfosuccinate, monothioglycerol, thiosorbitol, and ethylenediamine tetracetic acid. The ophthalmic formulation may be isotonic. The ophthalmic formulation may also include a surfactant or a stabilizer. Surfactants include Carbopol®. Stabilizers include sodium bisulfite, sodium metabisulfite and sodium thiosulfate.

The formulation may include an effective amount of a permeation enhancer that facilitates penetration of the formulation components through cell membranes, tissues, and extra-cellular matrices, including the cornea. The formulation may penetrate the surface of the eye to the conjunctiva. The “effective amount” of the permeation enhancer represents a concentration that is sufficient to provide a measurable increase in penetration of one or more of the formulation components through membranes, tissues, and extracellular matrices as just described. Suitable permeation enhancers include, by way of example, methylsulfonylmethane (MSM; also referred to as methyl sulfone), combinations of MSM with dimethylsulfoxide (DMSO), or a combination of MSM and, in a less preferred embodiment, DMSO, with MSM particularly preferred.

Kits and Packages

Provided herein are also kits and packages that include a compound of the disclosure, a retinal imaging device, and optionally suitable packaging. In one embodiment, a kit further includes instructions for use.

The retinal imaging device may include lens(es) and image sensors (thus forming a suitable retina scanner) for detecting a signal emitted. In some embodiments, the retinal imaging device detects a fluorescent signal. In some embodiments, the retinal imaging device further includes a laser light source which can be used to activate the fluorescent signal.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Imaging of Systemic Amyloid Deposits In Vivo

Three mice are bred and aged for imaging. The mice are investigated for live imaging of amyloid accumulation. A compound described herein is applied to the surface of the eye (Formulation: 200 μL of a 10 mg/mL solution of a compound described herein in 20% DMSO/80% propylene glycol), which allows for the visualization of TTR.

An optical imaging system modeled on a Zeiss Stemi 2000-C microscope is used. Input light is delivered near the axis of the microscope. Fluorescence excitation or far-red input is shaped spectrally with interference filters. Fluorescence emission bandpass filters are placed proximal to the integrated camera. The input source is based on a short arc, continuous xenon lamp, positioned adjacent to a spherical mirror that captures part of the energy and condenses it into the small diameter fiber optic cable (1.4 mm diameter NA about 0.5). The spectral output of the lamp provides a relatively uniform broad band light source from 400 to 700 nm with a power of approximately 1 mWatts/cm². The optical train shapes the spot size of the beam to avoid overfilling the pupil and provide efficient energy transfer of light from the illuminator to the mouse pupil. The outcome of the RIS design optical train results in a narrow beam focused onto the dilated pupil plane with an adjustable spot size ranging from 1 to 2 mm through modulation of just one optical element.

Fluorescence images of the conjunctiva are obtained and analyzed. A compound described herein fluoresces in contact with TTR protein aggregates, indicating that such compounds can be used for in vivo retina imaging as an indicator of systemic amyloidosis.

### Example 2: TTR Aggregate Preparations

Several aggregated TTR samples were prepared using different conditions as described below.
Aggregate A was prepared by resuspending TTR in 50 mM glycine buffer at a pH of about 3.6 and further incubating the same for 24 hours at room temperature.
Aggregate B1 was prepared by resuspending TTR in water at a pH of about 7.4, and further incubating the same for 7 days with stirring (200 rpm) maintained at 37° C.
Aggregate B2 was prepared by resuspending TTR in water at a pH of about 7.4, and further incubating the same for 14 days with stirring (200 rpm) maintained at 37° C.

### Example 3: Spectroscopic Properties of Compound I with TTR Aggregates

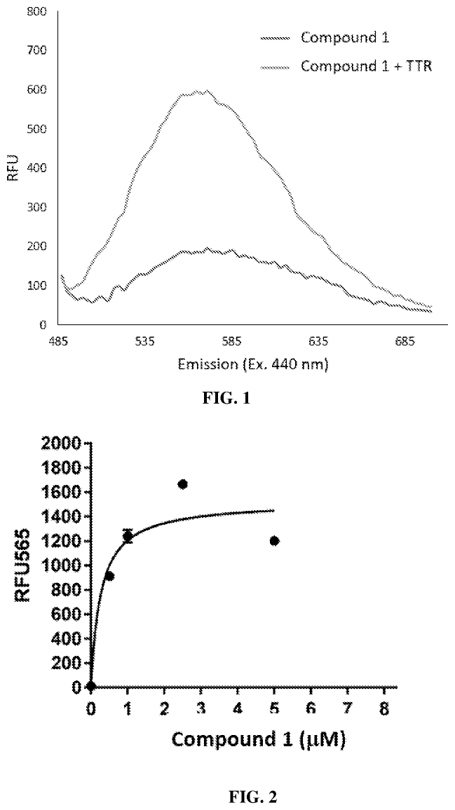

Compound 1 (4 μM) was prepared with or without Aggregate B1 in 1× phosphate-buffered saline (PBS). The absorption, fluorescence, and excitation spectra were taken for both samples. The excitation wavelength was optimized to be 440 nm. The emission spectra thus received were adjusted against a blank solvent control to obtain relative fluorescence intensity (RFI) as a function of wavelengths. As illustrated in FIG. 1, RFI of Compound 1 increased by a factor of about 3.2 in presence of Aggregate B1.

Further, fluorescence spectra were taken on mixtures of Compound I (4 μM) with Aggregate A, B1, or B2, respectively, each at the concentration of 5 μM, at the emission wavelength of 565 nm, when excited at 440 nm. The increases in RFI over Compound 1 alone were calculated and are illustrated in Table 1:

TABLE 1
Fluorescence Intensity Increase of Compound
1 in Presence of TTR Aggregates

| | Fluorescence fold increase |
|---|---|
| Compound 1 + Aggregate A | 4.6 |
| Compound 1 + Aggregate B1 | 2.3 |
| Compound 1 + Aggregate B2 | 2.5 |

### Example 4: Binding Affinity for Compound 1 with Aggregated TTR

A solution of DMSO/PBS (6 μL DMSO in 144 μL PBS, pH 7.4) was prepared as a blank control. Further, a 250 μM solution of Compound 1 in DMSO was prepared. The following samples were then prepared in triplicate while keeping a constant 5 μM TTR concentration in 4% DMSO/PBS:

TABLE 2
Samples Analyzed for Fluorescence
for Binding Constant Determination

| Final Compound | 250 μM | 100 uM | DMSO | PBS |
|---|---|---|---|---|
| Conc. (μM) | Compound (μL) | TTR (μL) | (μL) | (μL) |
| 10 | 6 | 7.5 | 0 | 136.5 |
| 5 | 3 | 7.5 | 3 | 136.5 |
| 2.5 | 1.5 | 7.5 | 4.5 | 136.5 |
| 1 | 0.6 | 7.5 | 5.3 | 136.5 |
| 0.5 | 0.3 | 7.5 | 5.7 | 136.5 |
| 0 | 0 | 7.5 | 6 | 136.5 |

Emission spectra were taken for each sample and adjusted against the blank over the range of 445-700 nm. RFI at λmax(em) was determined for each sample. RFI was then plotted against the increasing concentration of Compound 1 over the range of 0 to 10 μM using a one site specific binding algorithm. The binding affinity (K_{d}) was determined to be 0.3+/−0.1 μM. This is illustrated in FIG. 2.

### Example 5: Staining of ATTR Cadaver Eye

The following protocol was adopted for staining Formalin-Fixed Paraffin Embedded (FFPE) tissues:
1. Deparaffinization and hydration of tissue sections:
Pre-heat @ 60° C. 1 hour
Place slides in holders and treat with the clearing agent xylene (paraffin solvent) and a series of graded ethanol (EtOH) as follows:
   i. 100% xylene—5 min
   ii. 100% xylene—5 min
   iii. 50%/50% xylene/100% EtOH—3 min
   iv. 100% EtOH—3 min
   v. 95% EtOH—3 min
   vi. 70% EtOH—3 min
   vii. 50% EtOH—3 min
   viii. Water—2×3 min.
2. Antigen retrieval: incubate in 99% formic acid for 5 minutes.
3. Wash with distilled water for 5 minutes. Repeat this step two times.
4. Pre-heat 10 mM citrate buffer pH 6.0 to boiling.
5. Place slides in staining chamber with heated citrate buffer for 20 minutes.
6. Cool in water bath. Wash with distilled water for 5 minutes. Repeat this step two times.
7. Equilibrate in 1× PBS for 15 minutes.
8. Block in 5% Normal goat serum (NGS) in PBST (PBS with Tween 20) for 1 hour room temperature (RT).
9. Incubate in primary antibody O/N at 4° C. (2.5% NGS in PBST).
10. Wash tissues 3×10 minutes in PBST wash buffer.
11. Incubate in secondary antibody (1:500 in PBST) for 1 hour at RT—keep in dark from this point on.
12. Wash tissues 3×10 minutes in PBST.
13. Stain with compound (60 μM) for 30 minutes at RT.
   Let compound warm up to RT (˜30 min).
   Dissolve 5 mg of compound in 3.75 ml DMSO—keep in dark.
   Dilute 100 μl of compound solution in 5 ml PBS.
14. Wash tissues 3×10 minutes in PBST.
15. Stain nucleus with Hoechst (2:1000 from 1 mg/ml dilution in PBS) for 10 minutes.
16. Wash tissues 3×10 minutes in PBST.
17. Mount tissue using Prolong Glass mounting media, secure with clips and let dry ON.
18. Seal edges with nail polish.

### Example 6: Binding of Compound 1 to TTR in Human Tissues

Amydis retinal tracers are utilized to mark TTR aggregates in the eye for non-invasive detection using standard ocular imaging equipment, and thus facilitate diagnosis and monitoring of ATTR.

Sections from tissues of vitreous amyloid with known TTR mutation (paraffin embedded tissue blocks) and an autopsy eyeball with vitreous amyloid (paraffin embedded organ) are respectively stained with hematoxylin and eosin (H&E) and Congo red to mark regions with amyloidosis. Adjacent sections are co-stained with Compound 1 and an antibody specific for TTR using the optimized protocol described in Example 5. Immunofluorescent images are collected and compared. Regions of hyperfluorescence from Compound 1 are determined to correspond to areas of amyloidosis defined using Congo Red and H&E stains. It is contemplated that Compound 1 binds to TTR deposits in human tissues and undergoes an increase in fluorescence emission in a manner similar to its increase in fluorescence in vitro in the presence of aggregated TTR as described in earlier examples.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for determining whether a subject has systemic amyloidosis, comprising administering to an eye of the subject a compound of formula I, or a pharmaceutically acceptable salt thereof, and detecting the presence or absence of a misfolded or aggregated protein: 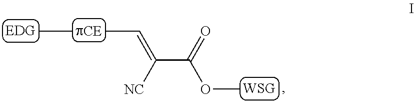
wherein
EDG is:
R¹-substituted or unsubstituted alkyl, R¹-substituted or unsubstituted cycloalkyl, R¹-substituted or unsubstituted heteroalkyl, R¹-substituted or unsubstituted heterocycloalkyl, R¹-substituted or unsubstituted aryl, R¹-substituted or unsubstituted heteroaryl, OR²NR⁴C(O)R³, —NR⁴R⁵, —SR⁶, or PR⁷R⁸,
wherein
R¹ is halogen, —OR⁹, —NR¹⁰R¹¹, unsubstituted alkyl, unsubstituted heteroalkyl, unsubstituted cycloalkyl, unsubstituted heterocycloalkyl, unsubstituted aryl, or unsubstituted heteroaryl;
R², R³, R⁴, R⁵, R⁶, R⁷ and R⁸ are independently hydrogen, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl or R¹²-substituted or unsubstituted heteroaryl, wherein R⁴ and R⁵ are optionally joined together to form an R¹²-substituted or unsubstituted heterocycloalkyl, or R¹²-substituted or unsubstituted heteroaryl;
R⁹ and R¹⁰ are independently hydrogen, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl, or R¹²-substituted or unsubstituted heteroaryl, wherein R¹⁰ and are optionally joined together to form an R¹²-substituted or unsubstituted heterocycloalkyl, or R¹²-substituted or unsubstituted heteroaryl;
R¹² is halogen, —OR¹³, —NR¹⁴R¹⁵, R¹⁶-substituted or unsubstituted alkyl, R¹⁶-substituted or unsubstituted heteroalkyl, R¹⁶-substituted or unsubstituted cycloalkyl, R¹⁶-substituted or unsubstituted heterocycloalkyl, R¹⁶-substituted or unsubstituted aryl, or R¹⁶-substituted or unsubstituted heteroaryl;
R¹³, R¹⁴ and R¹⁵ are independently hydrogen or unsubstituted alkyl; and
R¹⁶ is unsubstituted alkyl, unsubstituted heteroalkyl, unsubstituted cycloalkyl, unsubstituted heterocycloalkyl, unsubstituted aryl, or unsubstituted heteroaryl;
wherein
πCE has the formula:
-L¹-(A¹)_{q}-L²-(A²)ᵣ-L³- or-L¹-(A)_{q}-L⁴-A³-L²-(A²)ᵣ-L³-,
wherein
q and r are independently 0 or 1, wherein at least one of q or r is 1;
A¹, A² and A³ are independently R¹⁷-substituted or unsubstituted arylene or R⁷-substituted or unsubstituted heteroarylene;
L¹, L², L³ and L⁴ are independently a bond or a linking group having the formula: 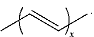
wherein x is an integer from 1 to 50;
R¹⁷ is halogen, —OR¹⁸, —NR¹⁹R²⁰, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl;
R¹⁸, R¹⁹ and R²⁰ are independently hydrogen, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl;
R²¹ is halogen, —OR²², —NR²³R²⁴, unsubstituted alkyl, unsubstituted heteroalkyl, unsubstituted cycloalkyl, unsubstituted heterocycloalkyl, unsubstituted aryl, or unsubstituted heteroaryl;
R²², R²³ and R²⁴ are independently hydrogen or unsubstituted alkyl; and
wherein
WSG is R²⁵-substituted or unsubstituted alkyl, R²⁵-substituted or unsubstituted heteroalkyl, R²⁵-substituted or unsubstituted cycloalkyl, R²⁵-substituted or unsubstituted heterocycloalkyl, R²⁵-substituted or unsubstituted aryl, R²⁵-substituted or unsubstituted heteroaryl;
wherein
R²⁵ is halogen, —OR²⁶, —NR²⁷R²⁸, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl;
R⁶, R²⁷ and R²⁸ are independently hydrogen, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl, wherein R²⁷ and R²⁹ are optionally joined together to form an R²⁹-substituted or unsubstituted heterocycloalkyl, or R²⁹-substituted or unsubstituted heteroaryl;
R²⁹ is halogen, —OR³⁰, —NR³¹R³², unsubstituted alkyl, unsubstituted heteroalkyl, unsubstituted cycloalkyl, unsubstituted heterocycloalkyl, unsubstituted aryl, or unsubstituted heteroaryl; and
R³⁰, R³¹ and R³² are independently hydrogen or unsubstituted alkyl; provided that the misfolded or aggregated protein is not amyloid beta peptide.

2. The method of claim 1, wherein the compound is 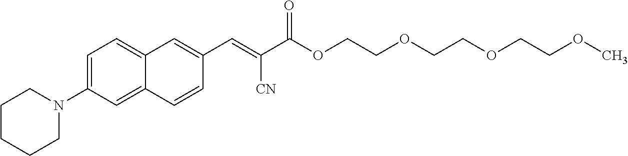 [SMILES: COCCOCCOCCOC(=O)/C(C#N)=C/c1ccc2cc(N3CCCCC3)ccc2c1]
or a pharmaceutically acceptable salt thereof.

3. The method of claim 1 or claim 2, wherein the misfolded or aggregated protein is transthyretin.

4. The method of any of the preceding claims, wherein the systemic amyloidosis is familial amyloidotic polyneuropathy (FAP), ATTR amyloidosis, TTR cardiac amyloidosis, TTR amyloid cardiomyopathy (ATTR-CM), familial or hereditary ATTR amyloidosis (ATTRv or ATTRm), senile systemic amyloidosis (SSA or ATTRwt), AL amyloidosis, AA (serum amyloid A amyloidosis), AA amyloidosis resulting from inflammation due to infection, rheumatological disease, autoinflammatory and autoimmune diseases whether acquired or hereditary, cancer or neoplasms, Fibrinogen alpha-chain amyloidosis, Apolipoprotein A-1 and A-2 Amyloidoses, Gelsolin amyloidosis, Ab2M (beta2 macroblobulin) amyloidosis, ALECT2 (leukocyte chemotactic factor 2) Amyloidosis, AApoAIV (Apolipoprotein AIV) amyloidosis, AApoCII (Apolipoprotein CII) amyloidosis, AApoCIII (Apolipoprotein CIII) amyloidosis, ALys (lysozyme) amyloidosis.

5. The method of any of the preceding claims, wherein administration is topical to a surface of the eye.

6. The method of any of the preceding claims, wherein the administration does not comprise administration directly to the interior of the eye.

7. The method of any of the preceding claims, wherein the administration does not comprise injection.

8. The method of any of the preceding claims, wherein contacting the compound with the misfolded or aggregated protein, upon activation by a light, causes emission of detectable signal.

9. The method of claim 8, wherein the detectable signal is a fluorescent signal.

10. The method of any of claims 3 to 9, wherein the misfolded or aggregated transthyretin protein is wild type.

11. The method of any of claims 3 to 9, wherein the misfolded or aggregated transthyretin protein is mutated.

12. A method for diagnosing systemic amyloidosis, comprising administering to an eye of a subject in need thereof a compound of formula I, or a pharmaceutically acceptable salt thereof, and detecting the presence or absence of a misfolded or aggregated transthyretin protein: 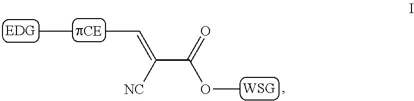
wherein
EDG is:
R¹-substituted or unsubstituted alkyl, R¹-substituted or unsubstituted cycloalkyl, R¹-substituted or unsubstituted heteroalkyl, R¹-substituted or unsubstituted heterocycloalkyl, R¹-substituted or unsubstituted aryl, R¹-substituted or unsubstituted heteroaryl, OR²NR⁴C(O)R³, —NR⁴R⁵, —SR⁶, or PR⁷R⁸,
wherein
R¹ is halogen, —OR⁹, —NR¹⁰R¹¹, unsubstituted alkyl, unsubstituted heteroalkyl, unsubstituted cycloalkyl, unsubstituted heterocycloalkyl, unsubstituted aryl, or unsubstituted heteroaryl;
R², R³, R⁴, R⁵, R⁶, R⁷ and R⁸ are independently hydrogen, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl or R¹²-substituted or unsubstituted heteroaryl, wherein R⁴ and R⁵ are optionally joined together to form an R¹²-substituted or unsubstituted heterocycloalkyl, or R¹²-substituted or unsubstituted heteroaryl;
R⁹ and R¹⁰ are independently hydrogen, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl, or R¹²-substituted or unsubstituted heteroaryl, wherein R¹⁰ and are optionally joined together to form an R¹²-substituted or unsubstituted heterocycloalkyl, or R¹²-substituted or unsubstituted heteroaryl;
R¹² is halogen, —OR¹³, —NR¹⁴R¹⁵, R¹⁶-substituted or unsubstituted alkyl, R¹⁶-substituted or unsubstituted heteroalkyl, R¹⁶-substituted or unsubstituted cycloalkyl, R¹⁶-substituted or unsubstituted heterocycloalkyl, R¹⁶-substituted or unsubstituted aryl, or R¹⁶-substituted or unsubstituted heteroaryl;
R¹³, R¹⁴ and R¹⁵ are independently hydrogen or unsubstituted alkyl; and
R¹⁶ is unsubstituted alkyl, unsubstituted heteroalkyl, unsubstituted cycloalkyl, unsubstituted heterocycloalkyl, unsubstituted aryl, or unsubstituted heteroaryl;
wherein
πCE has the formula:
-L¹-(A¹)_{q}-L²-(A²)ᵣ-L³- or-L¹-(A)_{q}-L⁴-A³-L²-(A²)ᵣ-L³-,
wherein
q and r are independently 0 or 1, wherein at least one of q or r is 1;
A¹, A² and A³ are independently R¹⁷-substituted or unsubstituted arylene or R¹⁷-substituted or unsubstituted heteroarylene;
L¹, L², L³ and L⁴ are independently a bond or a linking group having the formula: 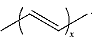
wherein x is an integer from 1 to 50;
R¹⁷ is halogen, —OR¹⁸, —NR¹⁹R²⁰, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl;
R¹⁸, R¹⁹ and R²⁰ are independently hydrogen, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl;
R²¹ is halogen, —OR²², —NR²³R²⁴, unsubstituted alkyl, unsubstituted heteroalkyl, unsubstituted cycloalkyl, unsubstituted heterocycloalkyl, unsubstituted aryl, or unsubstituted heteroaryl;
R²², R²³ and R²⁴ are independently hydrogen or unsubstituted alkyl; and
wherein
WSG is R²⁵-substituted or unsubstituted alkyl, R²⁵-substituted or unsubstituted heteroalkyl, R²⁵-substituted or unsubstituted cycloalkyl, R²⁵-substituted or unsubstituted heterocycloalkyl, R²⁵-substituted or unsubstituted aryl, R²⁵-substituted or unsubstituted heteroaryl;
wherein
R²⁵ is halogen, —OR²⁶, —NR²⁷R²⁸, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl;
R²⁶, R²⁷ and R²⁸ are independently hydrogen, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl, wherein R²⁷ and R²⁸ are optionally joined together to form an R⁹²-substituted or unsubstituted heterocycloalkyl, or R²⁹-substituted or unsubstituted heteroaryl;
R²⁹ is halogen, —OR³⁰, —NR³¹R³², unsubstituted alkyl, unsubstituted heteroalkyl, unsubstituted cycloalkyl, unsubstituted heterocycloalkyl, unsubstituted aryl, or unsubstituted heteroaryl; and
R³⁰, R³¹ and R³² are independently hydrogen or unsubstituted alkyl.

13. The method of claim 12, wherein the compound is 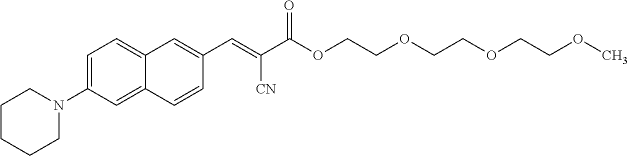 [SMILES: COCCOCCOCCOC(=O)/C(C#N)=C/c1ccc2cc(N3CCCCC3)ccc2c1]
or a pharmaceutically acceptable salt thereof.

14. The method of claim 12 or claim 13, wherein the systemic amyloidosis is familial amyloidotic polyneuropathy (FAP), ATTR amyloidosis, TTR cardiac amyloidosis, TTR amyloid cardiomyopathy (ATTR-CM), familial or hereditary ATTR amyloidosis (ATTRv or ATTRm), senile systemic amyloidosis (SSA or ATTRwt), AL amyloidosis, AA (serum amyloid A amyloidosis) AA amyloidosis resulting from inflammation due to infection, rheumatological disease, autoinflammatory and autoimmune diseases whether acquired or hereditary, cancer or neoplasms, Fibrinogen alpha-chain amyloidosis, Apolipoprotein A-1 and A-2 Amyloidoses, Gelsolin amyloidosis, Ab2M (beta2 macroblobulin) amyloidosis, ALECT2 (leukocyte chemotactic factor 2) Amyloidosis, AApoAIV (Apolipoprotein AIV) amyloidosis, AApoCII (Apolipoprotein CII) amyloidosis, AApoCIII (Apolipoprotein CIII) amyloidosis, ALys (lysozyme) amyloidosis.

15. The method of any of claim 12 to 14, wherein administration is topical.

16. The method of any of claim 12 to 15, whereby the compound reaches conjunctiva of the eye.

17. The method of any of claims 12 to 16, wherein the administration does not comprise administration to the interior of the eye.

18. The method of any of the preceding claims, wherein the administration does not comprise injection.

19. A method for preparing a patient for diagnosis of systemic amyloidosis, comprising topically administering to an eye of the patient a compound of formula I: 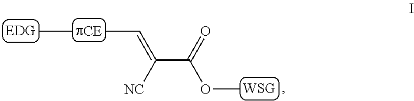
wherein
EDG is:
R¹-substituted or unsubstituted alkyl, R¹-substituted or unsubstituted cycloalkyl, R¹-substituted or unsubstituted heteroalkyl, R¹-substituted or unsubstituted heterocycloalkyl, R¹-substituted or unsubstituted aryl, R¹-substituted or unsubstituted heteroaryl, OR²NR⁴C(O)R³, —NR⁴R⁵, —SR⁶, or PR⁷R⁸,
wherein
R¹ is halogen, —OR⁹, —NR¹⁰R¹¹, unsubstituted alkyl, unsubstituted heteroalkyl, unsubstituted cycloalkyl, unsubstituted heterocycloalkyl, unsubstituted aryl, or unsubstituted heteroaryl;
R², R³, R⁴, R⁵, R⁶, R⁷ and R⁸ are independently hydrogen, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl or R¹²-substituted or unsubstituted heteroaryl, wherein R⁴ and R⁵ are optionally joined together to form an R¹²-substituted or unsubstituted heterocycloalkyl, or R¹²-substituted or unsubstituted heteroaryl;
R⁹ and R¹⁰ are independently hydrogen, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl, or R¹²-substituted or unsubstituted heteroaryl, wherein R¹⁰ and are optionally joined together to form an R¹²-substituted or unsubstituted heterocycloalkyl, or R¹²-substituted or unsubstituted heteroaryl;
R¹² is halogen, —OR¹³, —NR¹⁴R¹⁵, R¹⁶-substituted or unsubstituted alkyl, R¹⁶-substituted or unsubstituted heteroalkyl, R¹⁶-substituted or unsubstituted cycloalkyl, R¹⁶-substituted or unsubstituted heterocycloalkyl, R¹⁶-substituted or unsubstituted aryl, or R¹⁶-substituted or unsubstituted heteroaryl;
R¹³, R¹⁴ and R¹⁵ are independently hydrogen or unsubstituted alkyl; and
R¹⁶ is unsubstituted alkyl, unsubstituted heteroalkyl, unsubstituted cycloalkyl, unsubstituted heterocycloalkyl, unsubstituted aryl, or unsubstituted heteroaryl;
wherein
πCE has the formula:
-L¹-(A¹)_{q}-L²-(A²)ᵣ-L³- or-L¹-(A)_{q}-L⁴-A³-L²-(A²)ᵣ-L³-,
wherein
q and r are independently 0 or 1, wherein at least one of q or r is 1;
A¹, A² and A³ are independently R¹⁷-substituted or unsubstituted arylene or R¹⁷-substituted or unsubstituted heteroarylene;
L¹, L², L³ and L⁴ are independently a bond or a linking group having the formula: 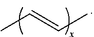
wherein x is an integer from 1 to 50;
R¹⁷ is halogen, —OR¹⁸, —NR¹⁹R²⁰, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl;
R¹⁸, R¹⁹ and R²⁰ are independently hydrogen, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl;
R²¹ is halogen, —OR²², —NR²³R²⁴, unsubstituted alkyl, unsubstituted heteroalkyl, unsubstituted cycloalkyl, unsubstituted heterocycloalkyl, unsubstituted aryl, or unsubstituted heteroaryl;
R²², R²³ and R²⁴ are independently hydrogen or unsubstituted alkyl; and
wherein
WSG is R²⁵-substituted or unsubstituted alkyl, R²⁵-substituted or unsubstituted heteroalkyl, R²⁵-substituted or unsubstituted cycloalkyl, R²⁵-substituted or unsubstituted heterocycloalkyl, R²⁵-substituted or unsubstituted aryl, R²⁵-substituted or unsubstituted heteroaryl;
wherein
R²⁵ is halogen, —OR²⁶, —NR²⁷R²⁸, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl;
R²⁶, R²⁷ and R²⁸ are independently hydrogen, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl, wherein R²⁷ and R²⁸ are optionally joined together to form an R²⁹-substituted or unsubstituted heterocycloalkyl, or R²⁹-substituted or unsubstituted heteroaryl;
R²⁹ is halogen, —OR³⁰, —NR³¹R³², unsubstituted alkyl, unsubstituted heteroalkyl, unsubstituted cycloalkyl, unsubstituted heterocycloalkyl, unsubstituted aryl, or unsubstituted heteroaryl; and
R³⁰, R³¹ and R³² are independently hydrogen or unsubstituted alkyl.

20. The method of claim 19, further comprising detecting the binding of the compound to a misfolded or aggregated protein.

21. The method of claim 20, wherein the misfolded or aggregated protein is transthyretin.

22. The method of claim 19, whereby the compound is delivered to conjunctiva of the eye.
